(19) United States
(12) Patent Application Publication
(10) Pub. No.: US 2013/0006337 A1
(43) Pub. Date: Jan. 3, 2013

(54) CHEMICAL WARMING DEVICE AND METHOD FOR THE TREATMENT OF VIRUSES SUCH AS HERPES

(76) Inventor: Americo Fernandes, Winnipeg (CA)

(21) Appl. No.: 13/577,214

(22) PCT Filed: Jan. 23, 2011
(86) PCT No.: PCT/IB2011/000223

§ 371 Date: Aug. 3, 2012

Related U.S. Application Data
(60) Provisional application No. 61/301,200, filed on Feb. 4, 2010.

(51) Int. Cl. A61F 7/08

(52) U.S. Cl. 607/109

(57) ABSTRACT

The present invention provides a new, practical, and inexpensive approach to labial herpes heat therapy. Heat is produced by the chemical reaction of elements contained in an inexpensive, portable and disposable air-activated chemical warming device that is small enough to be applied to an affected lip area. For example, a person can store the warming device at home and work, or simply carry a warmer in a purse or wallet. The warming devices can have a long shelf life.

### TECHNICAL FIELD

This invention relates to disease therapies and, in particular, to chemical warming devices and methods for the treatment of viruses such as herpes.

### BACKGROUND ART

The present invention is described in connection with herpes labialis as an example application; the invention is also applicable to genital herpes, other types of herpes, and other viruses that are susceptible to thermal treatment.

Recurrent herpes labialis is a common and ubiquitous condition that occurs in patients infected with the herpes simplex virus (HSV). HSV type 1 (HSV-1) is associated with oral blisters around the mouth that are commonly known as “cold sores”. Over 75 million Americans suffer from cold sores every year. Although most patients with recurrent herpes labialis experience only a few outbreaks annually, a significant number of patients experience more frequent and severe recurrences, sometimes more often than once a month. The HSV lays dormant in the body until reactivated by sunlight, stress, menstruation, or trauma to the area. The formation of cold sores is generally preceded by prodromal symptoms including localized tingling, burning, and itching. A recurrence of herpes labialis develops and matures very rapidly, reaching maximum severity within 8-16 hours of onset, leaving little time to intervene with an episodic therapy. Although not life-threatening, each episode causes pain, discomfort, and temporary disfigurement of the affected patient. Painful and unslightly cold sores can linger up to ten days. Particularly with young patients, the psychological impact of frequent or severe recurrences can be especially troubling. At present, no cure exists for this troublesome condition.

If the patient recognizes the prodromal symptoms, the patient can self-medicate to reduce healing time and lesion pain of an episode. Episodic therapies include over-the-counter topical antiviral creams, such as acyclovir, docosanol, and penciclovir, and peroral antiviral agents, such as acyclovir, valaciclovir, and famciclovir. Some of these therapies are modestly effective at reducing the duration of signs and symptoms of the disease. However, the clinical efficacy of many of these therapies for herpes labialis has not been proven, the medications are generally expensive, and some topical creams cause undue skin irritation.

A number of heat therapy devices have been described in the literature that may be useful for prevention of formation of a cold sore lesion, if applied in the prodromal phase, or for shortening the duration of the lesion, if applied later in the phase.

U.S. Pub, No. 2006/0142750 to Da Silva et al., which is incorporated herein by reference, describes a hand-held heating device that can be used for the treatment of acne, warts, cold blisters, blemished skin, or fine wrinkles. The heating element of the device comprises a battery that drives a short pulse of current through a resistive heating element.

U.S. Pub. No. 2007/0100403 to Felice et al., which is incorporated herein by reference, describes a hand-held therapeutic device for treating cold sores and other viral or bacterially caused conditions. The device uses a battery to power a heat source, such as a light bulb, which can be programmed to turn on or off for particular periods of time and at particular intervals.

U.S. Pat. No. 7,137,979 to Conrad et al., which is incorporated herein by reference, describes methods and devices for the treatment of skin lesions resulting from bacterial, viral, or fungal infections. The device can deliver a controlled dose of thermal energy to the infected or irritated tissue and thereby speed the recovery process. The heater can include a cartridge heater, a lamp or light bulb, or a foil-type heater that is powered by a battery or electricity from a conventional wall outlet.

U.S. Pat. Nos. 7,537,605 and 7,637,930 to Li et al., which are incorporated herein by reference, describe methods and systems for thermally treating skin disease. The hand-held apparatus uses a battery to power a heating element.

At present, several commercial products exist that purport to provide preventive heat therapy for cold sores. These include interceptCS (http://www.interceptcs.com/) and Hotkiss (claimed as a trademark by its owner) (http://www.herpotherm.co.nz/) medical devices.

However, these devices and products are costly, inconvenient, unreliable, impractical battery-powered heating devices. Therefore, a need remains for a heat therapy device for the treatment of cold sores and other infections that can provide advantages such as being inexpensive, portable, disposable, or combinations thereof.

### DISCLOSURE OF INVENTION

This application is related to the following application, which is incorporated herein by reference: U.S. 61/301,200, filed Feb. 4, 2010.

The present invention provides a preventative heat therapy for conditions such as herpes. The invention is effective in preventing the cold sore virus from expressing itself in the “itching phase” of the cold sore process. Although the devices and methods described herein are useful for the treatment of cold sores, the devices and methods can also be used for other bacterial, fungal, or viral infections that are responsive to thermal therapy.

Some embodiments of the invention comprise an air-activated chemical warming device for the treatment of cold sores, comprising a pouch comprising iron powder, salt, water, activated charcoal, and an absorbent material in a permeable covering, wherein oxygen reacts with the iron powder to release heat when the pouch is exposed to air; and a heat application surface on the covering that is soft and smooth for contact to the skin of a patient. Such embodiments can further comprise an outer protective packaging that is impermeable to oxygen and water for containing the pouch to extend the shelf life of the pouch when not in use. The device can further comprise a surface lubricant, oil, or moisturizer on the heat application surface to improve the skin contact properties of the surface. The iron powder, salt, water, active charcoal, and absorbent material can be selected to heat the pouch to a therapeutic temperature in the range of 50° C. to 52° C.

The invention further provides a method for the treatment of conditions such as cold sores, comprising providing a pouch comprising iron powder, salt, water, activated charcoal, and an absorbent material in a permeable covering and a heat application surface on the covering; exposing the pouch to air wherein oxygen in the air reacts with the iron powder to release heat; and contacting the heat application surface to an affected area of a lip. The contacting can occur when the pouch has reached a temperature in the range of 50° C. to 52° C. The contacting preferably occurs during the prodromal phase of the cold sore process.

### BRIEF DESCRIPTION OF DRAWINGS

The accompanying drawings, which are incorporated in and form part of the specification, illustrate the present invention and, together with the description, describe the invention. In the drawings, like elements are referred to by like numbers.

FIG. 1 is a photograph of individually packaged treatment pouches of an air-activated chemical warmer for the treatment of cold sores.

FIG. 2 is a schematic illustration of a method for the treatment of cold sores.

### MODES FOR CARRYING OUT THE INVENTION AND INDUSTRIAL APPLICABILITY

The present invention is described in the context of various example embodiments and applications. The present invention provides a new, practical, and inexpensive approach to treatments such as labial herpes heat therapy. Heat is produced by the chemical reaction of components contained in an inexpensive, portable and disposable air-activated chemical warming device that is small enough to be applied to an affected lip area. For example, a person can store a warmer at home and work, or simply carry a warmer in a purse or wallet. The devices have a long shelf life (as opposed to batteries, which can lose charge over time). As long as the air-activated pouches of the warmer are sealed, they are “fully charged” and ready for use. Further, the warmers are inexpensive and compact compared to current devices and products. In particular, current devices are too bulky to be conveniently carried in a wallet or small purse.

Disposable hand warmers are commonly used to heat up cold hands or feet on demand. They are typically used during outdoor winter activities, such as hiking, hunting, or skiing, to keep extremities warm and assist insulated clothing. Types of commercially available hand warmers include air-activated chemical warmers, reusable crystallization-type warmers that rely on the exothermic crystallization of supersaturated solutions, lighter fluids that react with a platinum catalyst to release heat by oxidation, and battery-operated warmers that rely on electrically resistive heating.

Air-activated chemical warmers rely on an exothermic reaction of air with iron that, in essence, creates rust. Typically, when iron is exposed to air it oxidizes to form iron oxide. The spontaneous oxidation reaction of bulk iron is so slow that the heat released is not noticeable. However, when this reaction is sped up, the amount of heat is noticeable and usable as a hand warmer. A typical air-activated chemical warmer comprises a pouch containing iron powder, salt, water, activated charcoal, and an absorbent material. To extend the shelf life of the pouch, the pouch can be contained in an airtight outer protective package that is impermeable to oxygen and water. When the pouch is removed from the outer packaging, oxygen from the air permeates through the pouch's permeable covering. With salt and water present, the oxygen reacts with the iron powder to form iron oxide and release heat (i.e., 4Fe+302->2Fe203+heat). For example, the protective package can comprise an impermeable polymer, such as polyethylene. For example, the pouch covering can be a blended nonwoven material with specific permeability characteristics, such as cellulose. The salt acts as a catalyst to speed up the rate of reaction. For example, the salt can be sodium chloride. Water can facilitate oxidation. Activated carbon can help to evenly disperse the heat produced throughout the warmer and to absorb odors. The absorbent can help to retain moisture and promote diffusion of gas throughout the pouch by keeping the components from agglomerating. It also can act as an insulator, keeping the heat from dissipating too rapidly. The absorbent material can be a non-reactive insulator, such as vermiculite, pulverized wood, or a superabsorbent polymer, such as polyacrylate.

The temperature, the time that the pouch requires to heat up, and the length of time that the pouch remains heated can be varied by changing the raw materials in the pouch. The temperature attained is largely dependent on the rate of the oxidative reaction, which is controlled by the amount of iron and oxygen available to react. For example, the pouch will remain heated longer by increasing the amount of iron in the pouch. The warmer can last for hours. The reaction time, and therefore the time required to heat the pouch up, can be reduced by increasing the surface area of the iron (e.g., by finely dividing the powder) or by increasing the permeability of the pouch covering (e.g., increasing the permeability will admit more oxygen, allowing the reaction to occur more rapidly). The average hand warmer temperature is 48° C. to 65° C. Existing hand warmers are not suitable for treatment of cold sores, at least because they are not configured for convenient application and because they produce temperatures that are not precisely controlled as required to effectively treat cold sores.

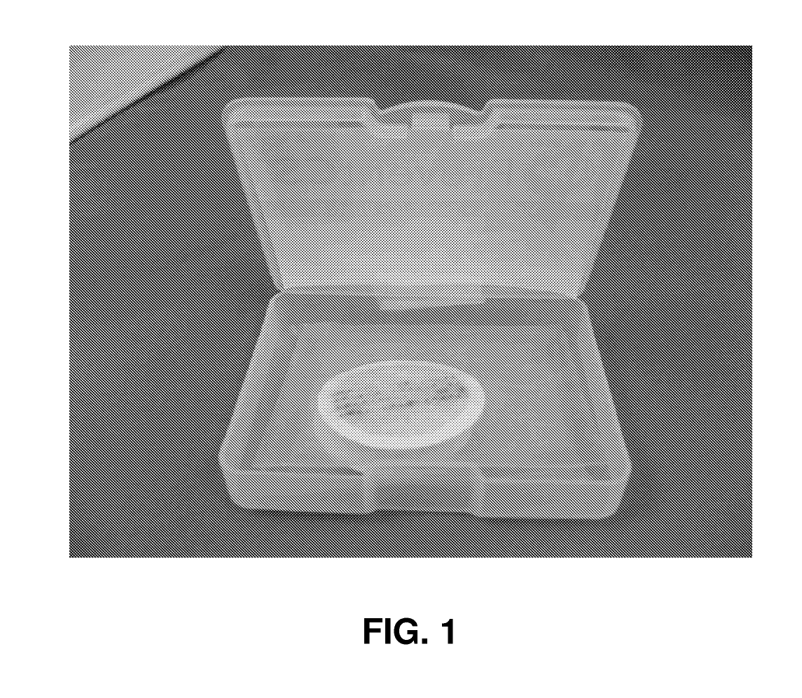

FIG. 1 is a photograph of individually packaged treatment pouches of an air-activated chemical warming device for the treatment of cold sores. For cold sores, the temperature reached and the duration of treatment is preferably high and long enough to aid in the prevention or recovery process, yet low enough to avoid burning the skin or causing excessive discomfort. A heat range of 50° C. to 52° C. is recommended by research as the ideal temperature for the prevention of labial herpes. Multiplication of the HSV is ideally stopped with a therapeutic temperature of 51° C. Therefore, the chemical components of the device are preferably precisely calibrated to provide an average temperature of 51° C. For example, the device can comprise 13 grams of iron powder, 0.5 grams of NaCl, and 2.5 grams of vermiculite in a pouch of suitable size. Further, the outer covering material of existing hand warmers is too rough and can irritate the lip or genital tissue during application. Some embodiments of the present invention use an outer heat application surface on the pouch that is smooth and soft, such as paper, textile, or plastic. This soft, smooth surface will minimize surface contact irritation of the lip or other tissue site. The heat application surface can be sized to cover the affected area of the lip or other tissue site. The heat application surface can also be treated with a surface lubricant, such as Vaseline or aloe lotion, oils, moisturizers such as Nivea, or other topical substances to improve the surface contact properties.

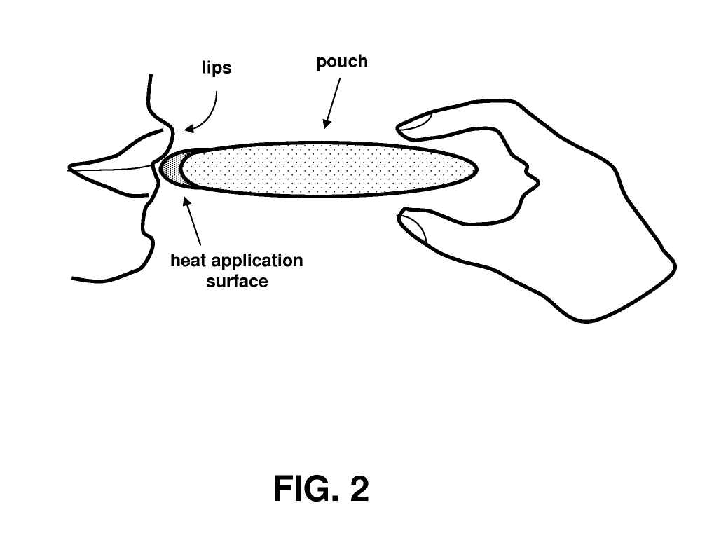

FIG. 2 is a schematic illustration of a method for the treatment of cold sores. The timing of heat treatment is important. The treatment is most effective if heat is applied at the beginning, prodromal phase of the cold sore process. Those who suffer from the virus can recognize the symptoms and apply the heat treatment at an optimum time. When the patient feels the tingling or itching of a cold sore, the outer protective package can be opened when ready to use. The inner pouch can be removed from the protective package without opening the inner pouch. The pouch can be shaken to mix and activate the contents. Fifteen to thirty minutes can be allowed for the warmer to heat up. The heat application surface of the pouch can then be placed to the pursed lips or other affected tissue site of the patient, for example, for three 30 second intervals separated by one minute between each application. Treatment can be safely repeated as long as the symptoms persist. After use, the pouch can be disposed of with regular garbage. The all natural ingredients will not harm the environment.

The present invention has been described as an air-activated chemical warming device and method for the treatment of cold sores. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1) An air-activated chemical warming device for the treatment of cold sores, comprising:
a. a pouch comprising iron powder, salt, water, activated charcoal, and an absorbent material in a permeable covering, wherein oxygen reacts with the iron powder to release heat when the pouch is exposed to air, and wherein the relative proportions of the constituents of the pouch provide an output temperature within the treatment range for cold sores; and
b. a heat application surface on the pouch that is amenable for effective therapeutic contact to the skin of a patient.

2) The device of claim 1, further comprising an air-tight protective packaging that is substantially impermeable to oxygen and water and configured to contain the pouch such that the iron is not exposed to excess oxygen while the pouch is in the protective packaging.

3) The device of claim 1, further comprising a surface lubricant, oil, or moisturizer on the heat application surface to improve the skin contact properties of the surface.

4) The device of claim 1, wherein the quantities of iron powder, salt, water, active charcoal, and absorbent material are selected to heat the pouch to a temperature in the range of 50° C. to 52° C.

5) A method for the treatment of cold sores, comprising:
a. providing a pouch as in claim 1,
b. exposing the pouch to air wherein oxygen in the air reacts with the iron powder to release heat, and
c. contacting the heat application surface to an affected area of an affected tissue site.

6) The method of claim 5, wherein the contacting occurs when the pouch has reached a temperature in the range of 50° C. to 52° C.

7) The method of claim 5, wherein the contacting occurs during the prodromal phase of the cold sore process.

8) The method of claim 5, further comprising storing the pouch in an air-tight protective package prior to exposing the pouch to air.
